(19) United States
(12) Plant Patent Application Publication
(10) Pub. No.: US 2003/0061647 P1
(43) Pub. Date: Mar. 27, 2003

(54) Forsythia plant named 'Courtacour'

(76) Inventor: M. Alain Cadic, Beaucouze (FR)

Correspondence Address: BURNS, DOANE, SWECKER & MATHIS, L.L.P., P.O. BOX 1404, Alexandria, VA, 22313-1404, US

(21) Appl. No.: 10/288,510

(22) Filed: Nov. 6, 2002

Related U.S. Application Data
(63) Continuation of application No. 09/607,278, filed on Jun. 30, 2000.

(51) Int. Cl. A01H 5/00

(52) U.S. Cl. PLT/230

(57) ABSTRACT

A new and distinct cultivar of Forsythia plant is provided that is a seedling formed following the induced mutation of the ‘Spring Glory’ cultivar (non-patented in the United States). Gamma radiation was utilized to create the mutation. The new cultivar can be readily distinguished from the parent cultivar by the presence of a very compact dwarf growth habit with exceptionally short internodes. Attractive bright yellow flowers are formed on an abundant basis. In view of the short internodes the blossoms well cover the relatively thick branches. Dark green foliage begins to appear during flowering. The winter hardiness is good. The plant is particularly well suited for growing in containers. The new cultivar also is well suited for growing as attractive ornamentation in the landscape.

### SUMMARY OF THE INVENTION

A new and distinct variety of Forsythia×intermedia plant is provided that is a mutation derived from the ‘Spring Glory’ cultivar (non-patented in the United States).

When creating the new cultivar of the present invention plants of the ‘Spring Glory’ cultivar were irradiated with gamma rays, seeds were formed thereon following open pollination, and the seedlings produced upon the planting of such seeds were observed. The irradiation and the study of the resulting seedlings were carried out at the Institut National De La Recherche Agronomique located at Angers, France.

It was found that a single plant observed following such irradiation and the planting of seeds possessed the characteristics of the Forsythia cultivar of the present invention. The characteristics of such new cultivar can be summarized as follows:

(a) exhibits a very compact dwarf upright spreading growth habit with exceptionally short internodes,

(b) forms attractive bright yellow flowers on an abundant basis,

(c) forms dark green foliage that begins to appear during flowering,

(d) exhibits good winter hardiness, and

(e) is well suited for growing in containers.

The new cultivar of the present invention well meets the needs of the horticultural industry and is particularly well suited for growing as attractive ornamentation. The bright yellow blossoms are formed in abundance in the springtime and in view of the short internodes well cover the relatively thick branches. Adjoining blossoms commonly touch and overlap each other. Even young plants are very floriferous.

The size of the plant at maturity is considerably smaller than that of the ‘Spring Glory’ cultivar. The new cultivar grows well in containers, tubs, and pots and can be grown on balconies and terraces. Alternatively, it can be grown to advantage in the landscape where an attractive smaller-sized ornamental plant is desired.

The new cultivar has been found to readily undergo asexual propagation by the use of softwood cuttings. Roots readily form on such cuttings during the summer. Such asexual propagation has been carried out at Angers, France, and elsewhere.

The new cultivar has been named the ‘Courtacour’ cultivar.

### BRIEF DESCRIPTION OF THE PHOTOGRAPH

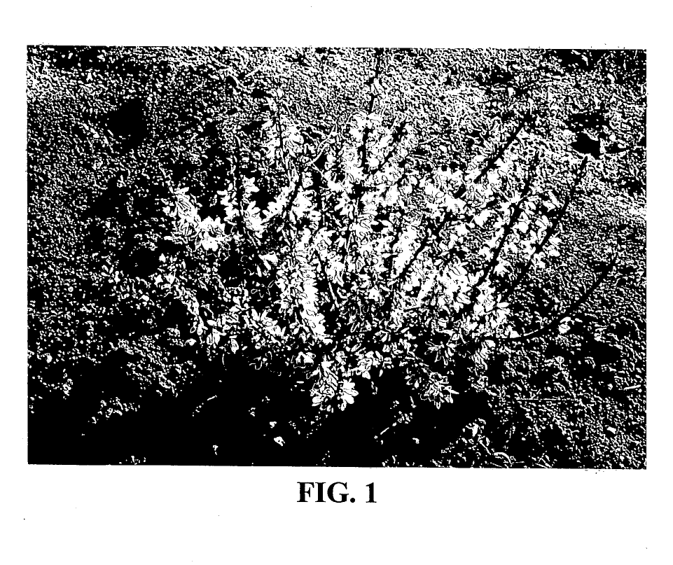

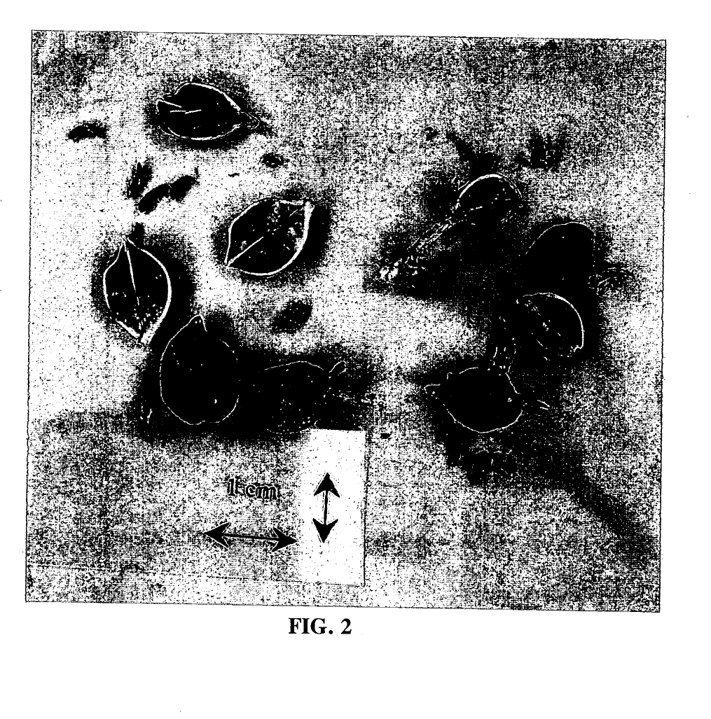

The accompanying FIG. 1 shows a typical plant of the new variety when blossoming during the springtime. The plant was being grown outside at Angers, France. The dwarf upright spreading growth habit and profusion of bright yellow flowers closely borne along branches are apparent.

### DETAILED DESCRIPTION

The following description is based on the observation of plants of the new cultivar growing outdoors at Angers, France. Such plants had been asexually reproduced through the use of softwood cuttings. The color terminology utilized in the description that follows is to be accorded its ordinary dictionary significance. Reference to the R.H.S. Colour Chart of The Royal Horticultural Society, London, England, sometimes is included.

Origin: Seedling produced following the induced mutation of the ‘Spring Glory’ cultivar (non-patented in the United States). The ‘Spring Glory’ cultivar was irradiated with gamma rays from cobalt 60.

Parentage: Seedling of ‘Spring Glory’.

Classification: Forsythia×intermetia, cv. ‘Courtacour’.

Plant:

Form.—well-branched flowering shrub.

Habit.—erect, upright, spreading, and very compact. The plant is considered to be a dwarf unlike its ‘Spring Glory’ parent.

Internode length.—exceptionally short, considerably shorter than that of the parent ‘Spring Glory’ cultivar. Typical internode lengths are approximately 20 to 40 mm.

Height.—approximately 0.5 to 0.8 m at maturity, and considerably shorter than the ‘Spring Glory’ cultivar. The ‘Spring Glory’ cultivar commonly will assume a height of approximately 1.5 m.

Width.—approximately 1 m at maturity.

Foliage:

Disposition.—opposite, and commonly begins to appear during flowering.

Configuration.—ovate and simple.

Color.—dark green.

Inflorescence:

Configuration.—more compact than those of the ‘Spring Glory’ cultivar.

Size.—medium.

Color.—bright yellow, Yellow Group 12A. This compares to Yellow Group 7A for the parent ‘Spring Glory’ cultivar.

Sepals.—green in coloration.

Petals.—elliptic in configuration, and shorter than those of ‘Spring Glory’.

Disposition.—closely spaced along the branch (as illustrated) in view of exceptionally short internode length, and are borne on short pedicels.

Anthers.—tend to extend above the stigma.

Time of flowering.—springtime, and approximately 15 days later than the ‘Spring Glory’ cultivar.

Petal drop.—medium tendency to detach.

Growing Conditions: Does well in full sun or semi-shade, and prefers soil that is not exceedingly dry.

Hardiness: Very good. Has withstood temperatures of −25° C. to −30° C.

Propagation: Can be readily propagated while utilizing softwood cuttings.

Usage: Attractive erect ornamental spring-flowering shrub for growing in pots, tubs or containers in small gardens, balconies and terraces. Alternatively can be grown in the landscape. Requires little care.

I claim:

1. A new and distinct Forsythia cultivar that is derived from the ‘Spring Glory’ (non-patented in the United States) cultivar having the following combination of characteristics:
(a) exhibits a very compact dwarf upright spreading growth habit with exceptionally short internodes,
(b) forms attractive bright yellow flowers on an abundant basis,
(c) forms dark green foliage that begins to appear during flowering,
(d) exhibits good winter hardiness, and
(e) is well suited for growing in containers;
substantially as shown and described.
